(19) United States
(12) Patent Application Publication
(10) Pub. No.: US 2013/0022655 A1
(43) Pub. Date: Jan. 24, 2013

(54) Metal Oxide Nanocomposites for UV Protection

(71) Applicants: Bernd Sachweh, Meckenheim (DE); Wieland Koban, Mannheim (DE); Wendell Wohlleben, Mannheim (DE); Wolfgang Peukert, Markt Schwaben (DE); Robin Klupp Taylor, Forchheim (DE); Monica Distaso, Uttenreuth (DE)

(72) Inventors: Bernd Sachweh, Meckenheim (DE); Wieland Koban, Mannheim (DE); Wendell Wohlleben, Mannheim (DE); Wolfgang Peukert, Markt Schwaben (DE); Robin Klupp Taylor, Forchheim (DE); Monica Distaso, Uttenreuth (DE)

(73) Assignee: BASF SE, Ludwigshafen (DE)

(21) Appl. No.: 13/509,665

(22) PCT Filed: Nov. 15, 2010
(86) PCT No.: PCT/EP10/67425

§ 371 Date: May 14, 2012

Related U.S. Application Data
(60) Provisional application No. 61/261,387, filed on Nov. 16, 2009.

(30) Foreign Application Priority Data
Nov. 16, 2009 (EP) 09176122.1
Nov. 23, 2009 (EP) 09176751.7

(51) Int. Cl. A61K 8/02; A61K 8/81; A61Q 17/04; B82Y 40/00; B82Y 99/00

(52) U.S. Cl. 424/401; 424/59; 977/773; 977/788; 977/926; 977/900

(57) ABSTRACT

The present invention relates to a method of protecting a substrate against ultraviolet (UV) irradiation by applying to the substrate metal oxide nanocomposite particles showing at the same time high transmittance of visible light and high absorbance of UV light.

The present invention relates to a method of protecting a substrate against ultraviolet (UV) irradiation by applying to the substrate metal oxide nanocomposite particles showing at the same time high transmittance of visible light and high absorbance of UV light.

One embodiment of this invention specifically relates to sunscreen/cosmetic compositions, having improved properties and comprising oil-in-water type emulsions (in a cosmetically acceptable vehicle or carrier) that contain, as photo-protective agents such metal oxide nanocomposite particles.

UV-absorbing metal oxide nanoparticles are known in applications as diverse as pigments, catalysts, antibacterial products and cosmetic sunscreens. In many of these applications it is particularly desirable that the scattering of visible light is very low whilst UV absorption is maintained. This is usually achieved in the art by providing nanoparticles of pure metal oxide with a sufficiently small particle size. Many inventions relating to the preparation of small metal oxide nanoparticles have been reported.

Sunscreen compositions are broadly classified into “chemical” (organic) or “physical” (inorganic) sunscreens depending on the nature of the active ingredient which acts to screen out UVA and UVB radiation.

Physical sunscreens typically consist of a dispersion of particles of inert inorganic compounds which preferentially absorb UV radiation and which may also scatter UV and visible radiation depending on the size of the particles, the wavelength of the UV radiation, and the difference in refractive index of the dispersed particles and the dispersion medium. It is well known e.g. in the cosmetics industry that certain metal oxides, including zinc oxide and titanium oxide, are effective physical UV screening agents. Zinc oxide in particular is known to have a high absorbance to UV radiation over virtually the entire spectrum of UVB (280-320 nm) and UVA (320-400 nm) radiation. The inclusion of zinc oxide as a physical UV absorber in sunscreens is known.

Physical sunscreens, particularly those containing zinc oxide, are sometimes preferable over chemical sunscreens because they are known to be UV stable and exhibit no known adverse effects associated with long-term contact with the skin.

The major limiting factor in the use of conventional physical UV screening agents is the tendency for sunscreen formulations including such physical UV screening agents to appear white on the skin due to excessive scattering of light from the particles contained within such sunscreen formulations. This results in low cosmetic acceptability and marketability of sunscreen formulations which rely on conventional physical UV screening agents alone.

In sunscreens containing physical UV screening agents the transparency decreases with increasing concentration of the physical sunscreen particles. This is because of an increased scattering of light by the particles, which causes a whitening effect in the layer of the sunscreen. Thus, for a given layer thickness there is typically a trade-off between the transparency of the layer and the concentration of physical screening agents in the layer. In known commercially available sunscreens the whitening effect limits the maximum concentration of physical UV screening agents, such as zinc oxide or titanium oxide, in sunscreens to values which are sometimes unable to provide adequate UVA/UVB protection. As a consequence, acceptable values of Sun Protection Factor (SPF) can sometimes only be achieved by adding chemical UV screening agents to the sunscreen.

The Sun Protection Factor (SPF) determined in vivo is a universal indicator of the efficacy of sunscreen products against sunburn.

An individual Sun Protection Factor (SPFi) value for a product is defined as the ratio of the Minimal Erythemal Dose on product protected skin (MEDp) to the Minimal Erythemal Dose on unprotected skin (MEDu) of the same subject:

SPFi=MEDi (protected skin)/MEDi (unprotected skin)=MEDpi/MEDui

The SPF for the product is the arithmetic mean of all valid individual SPFi values obtained from all subjects in the test, expressed to one decimal place.

As mentioned above, one of the main limitations of the use of physical UV screening agents in sunscreens is the problem of whiteness left on the skin after the sunscreen has been applied. If an image-conscious user of the sunscreen applies a thin layer of that sunscreen to avoid this whiteness effect, the effective SPF will be less than that measured in the standard tests due to the fact that any SPF rating is dependent on the thickness of the layer of sunscreen tested. Thus the SPF measured in an SPF test may not be obtained by the user in the actual usage of the product if they are concerned about avoiding whitening.

In recent years, there is a trend in the cosmetic sunscreen industry to develop and use sunscreen formulations containing zinc oxide of smaller and smaller particle size to reduce the whiteness and improve the transparency of sunscreen formulations. However, in addition to the challenges of manufacturing such small particles, postprocessing like e.g. stabilizing and dispersing, is significantly more complicated.

Many inventions relating to the preparation of small metal oxide nanoparticles have been reported. In addition to the formation of the metal oxide, a vital aspect of most recent developments is the stabilization of the particles against precipitation and/or aggregation, either during or after formation. This stabilization usually takes the form of a surface modification of the particles' surfaces with amphiphilic molecules or polymers and is supposed to provide the repulsive interactions between the particles needed to prevent coagulation. In applications such properties are essential in order to enable an e.g. pigment powder to be formed and later redispersed or to provide long-term stability to a liquid formulation. In the case of insufficient stabilization, random coagulation of particles will occur, resulting in decreased transparency of films and coatings formed from them.

It would appear that for best optical performance particles should be as small and as well-dispersed as possible. Many recent developments try to provide this by producing primary particles smaller than e.g. 50 nm and by stabilizing them against aggregation by various means. In many cases, however, aggregates of primary particles with a rather uncontrolled aggregate size are formed with generally undisclosed influence on the transparency.

US 2007/243145 and US 2008/0193759 describe the production of surface-modified metal oxide particles by low temperature aqueous processing of metal salts in the presence of vinylpyyrolidone copolymers.

US 2008/0254295 and US 2007/0218019 report the production of particles of surface modified metal oxide, metal hydroxide and/or metal oxyhydroxide or metal oxide being formed by heating aqueous metal salt solutions in the presence of polyaspartic acid. Powders formed from such dispersions were found to consist of aggregates of small nanocrystallites.

WO 2008/116790 describes the production of surface modified metal oxide particles with a typical size of 40 to 80 nm via treatment of metal salts in aqueous solution in the presence of a strong base and polyacrylate.

WO 2008/043790 describes the production of surface modified metal oxide particles via treatment of metal salts in aqueous solution in the presence of a non-ionic dispersant with 2 to 1000 ethylene oxide units.

DE 102005055079 describes the production of amorphous titanium dioxide particles by hydrolysis of titanium tetraalcoholate in aqueous solution in the presence of a polyethylene glycol stabilizer.

WO 2004/052327 describes the formation of dispersions of surface-coated zinc oxide nanoparticles in non-polar or low-polarity solvents by the treatment with surfactants with a carboxylic acid headgroup.

Yao, K. X. et al. (J. Phys. Chem. C., 111, 13301, 2007) describe Zinc Oxide nanocomposite spheres and the manufacture thereof.

It is one object of the present invention to provide metal oxide particles which show high absorbance of UV light as well as high transmission of visible light. At the same time, such particles should be easily dispersible and compositions containing such particles should be stable against coagulation. Furthermore, the particle size distribution should be substantially narrow.

It is another object of the present invention to provide UV absorbing material that effectively protects the substrates to which it is applied against UV irradiation and at the same time does not substantially alter the transparency of such substrates with respect to visible light.

It is another object of the present invention to provide a substantially visibly transparent topical sunscreen composition, which, when applied to the skin, does substantially not cause whitening of such skin.

It is another object of the present invention to provide a UV screening composition for polymer substrates that does not substantially alter the transparency of such substrates.

The terms “sunscreen” and “UV screening agents” throughout this specification in no way imply or suggest that 100% blockage of UV radiation occurs. These terms are merely used to describe the role of the agent or composition in reducing the extent to which UV radiation is able to access the substrate.

The present invention circumvents the problems associated with the manufacture and stabilization of very small particles. It was found that the transparency of a layer can be improved while maintaining the UV protection properties by using metal oxide composites. The particles according to this invention provide significantly improved optical properties when compared to respective metal oxide particles of comparable size known in the art.

According to one aspect of the present invention, there is provided a method of protecting an object against UV radiation comprising applying to said object an effective amount of a composition containing a metal oxide nanocomposite, said metal oxide nanocomposite

a) having a number average particle size in the range of from 80 nm to 400 nm, and

b) comprising at least one metal oxide and at least one polymer, and

c) being substantially in the form of interconnected metal oxide units dispersed in a matrix substantially consisting of the at least one polymer.

The term “effective amount” means an amount of the composition according to this invention the application of which to an object results in an increase of the SPF compared to the SPF of the untreated object.

Throughout this specification, the term “object” can mean anything that is to be protected against damages caused by UV irradiation. In one embodiment of the present invention, said object is the human skin. In another embodiment of the present invention, said object is an article at least partially consisting of UV sensitive plastics like for example articles made of UV sensitive thermoplastics.

The skilled person, like e.g. a sunscreen formulator, would be readily able to determine the effective amount, i.e. the weight percentage in compositions, of the physical UV screening agent required to achieve the desired level of UV protection.

The term “composition” is intended to cover any composition containing a metal oxide nanocomposite and at least one other ingredient.

In one embodiment of the present invention, the term “composition” is intended to cover a dispersion, an emulsion (either a cream or a lotion), a stick, a gel, a spray, a clear lotion, or a wipe or any other composition suitable for use in protecting skin against sun damage. The dispersion or emulsion may be a water-in-oil emulsion, or an oil-in water emulsion, or a multiple phase emulsion.

In one embodiment of the present invention, said composition is substantially visibly clear and transparent.

The term “metal oxide nanocomposite” means a plurality of particles having a number average particle size in the range of from 80 nm to 400 nm, such particles comprising at least one metal oxide and at least one polymer and said metal oxide being substantially in the form of interconnected metal oxide units dispersed within a matrix substantially consisting of the at least one polymer. The composite particles forming the metal oxide nanocomposite are referred to as “metal oxide nanocomposite particles” or simply “particles” throughout this specification.

The term “matrix” as used herein means the phase substantially consisting of the at least one polymer and surrounding the mostly interconnected metal oxide units. The at least one metal oxide is existent mainly in the form of aggregates of smaller units (grains). These aggregates and few non-aggregated smaller units are surrounded by the phase substantially consisting of the at least one polymer. In one embodiment of the present invention, the size of these smaller units (hereinafter also referred to as grains or subunits) forming the discontinuous phase is in the range of from 1 to 20 nm. In a preferred embodiment of the present invention, the size of these smaller units forming the discontinuous phase is in the range of from 3 to 10 nm. In another preferred embodiment of the present invention, the size of these subunits forming the discontinuous phase is in the range of from 3 to 8 nm. Although the sub-units are mainly interconnected to each other, the size described before refers to the size of the subunits as they can be distinguished from each other by visual inspection of the electron micrographs. Alternatively, the size may be determined by evaluating the broadening of the peaks in the diffraction pattern by applying the Scherrer equation to the most intense peak.

The skilled person is aware of appropriate methods to determine the particle size of objects in the sub-micrometer range. In one embodiment of the present invention, the number average particle size is determined by Scanning Electron Microscopy (SEM) or Transmission Electron Microscopy (TEM).

The number average particle size of the metal oxide nanocomposite, as determined by electron microscopy, is in the range of from 80 nm to 400 nm. In a preferred embodiment of this invention, the number average particle size of the metal oxide nanocomposite is in the range of from 100 to 400 nm.

The metal oxide nanocomposite particles of this invention may have different shapes, such shapes including disks, low aspect ratio prisms or half-prisms, low aspect ratio ellipsoids or half-ellipsoids, spheres or half-spheres.

In one preferred embodiment of this invention, the majority of the metal oxide nano-composite particles of this invention has a substantially ellipsoid form. In another preferred embodiment of this invention, the majority of the metal oxide nano-composite particles of this invention has a substantially spherical form.

The term “majority” means in one embodiment of this invention more than 50%, in another embodiment of this invention at least 80%, in still another embodiment of this invention at least 90% and in still another embodiment of this invention at least 98% of all metal oxide nanocomposite particles.

“A substantially spherical form” means that the aspect ratio, i.e. the ratio of the longest and shortest axis of the three-dimensional shape (longest axis / shortest axis) is in the range of from 1,3:1 to 1:1 (1:1 corresponds to a perfect sphere), preferably from 1,2:1 to 1:1, more preferably from 1,1:1 to 1:1.

Additionally, “substantially spherical form” means that the metal oxide nanocomposite particles' surface is not perfectly even and smooth but rough as can be seen from the electron micrographs.

Metal Oxide

According to the invention, the metal oxide is substantially in the form of interconnected metal oxide units dispersed in a matrix substantially consisting of the at least one polymer.

“Substantially in the form of interconnected metal oxide units” means, that the major part of the metal oxide is present in the form of interconnected metal oxide units. Preferably, at least 90 weight-% of the metal oxide are present in the form of interconnected metal oxide units. More preferably, at least 95 weight-% of the metal oxide are present in the form of interconnected metal oxide units. Still more preferably, at least 98 weight-% of the metal oxide are present in the form of interconnected metal oxide units.

“Interconnected” means, that the respective metal oxide unit directly touches at least one other metal oxide unit.

The metal oxide is preferably selected from the oxides of the metals selected from the group consisting of aluminum, magnesium, cerium, iron, manganese, cobalt, nickel, copper, titanium, zinc and zirconium.

In one embodiment of this invention, the metal oxide is preferably selected from the oxides of the metals selected from the group consisting of cerium, titanium, and zinc. In one preferred embodiment of this invention, the metal oxide is Zinc oxide.

Polymer

According to the invention, the metal oxide is substantially in the form of interconnected metal oxide units dispersed in a matrix substantially consisting of the at least one polymer.

“Substantially consisting of” means that the major part of the matrix consists of the at least one polymer. Preferably, at least 90 weight-% of the matrix consist of the at least one polymer. More preferably, at least 95 weight-% of the matrix consist of the at least one polymer. Still more preferably, at least 98 weight-% of the matrix consist of the at least one polymer.

The at least one polymer is selected from polymers being capable of forming coordinative interactions with the metal cations of the at least one metal oxide precursor. In a preferred embodiment of this invention, the at least one polymer is selected from polymers comprising, as polymerized units, monomers of formula I:

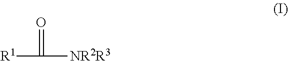

where R¹ is a group of the formula CH₂═CR⁴— where R⁴═H or C₁-C₄-alkyl and R² and R³, independently of one another, are H, alkyl, cycloalkyl, heterocycloalkyl, aryl or hetaryl or R² and R³ together with the nitrogen atom to which they are bonded are a five- to eight-membered nitrogen heterocycle or

R² is a group of the formula CH₂═CR⁴— and R¹ and R³, independently of one another, are H, alkyl, cycloalkyl, heterocycloalkyl, aryl or hetaryl or R¹ and R³ together with the amide group to which they are bonded are a lactam having 5 to 8 ring atoms.

Preferred monomers of formula (I) are N-vinyllactams and derivatives thereof. Suitable monomers of formula (I) are e.g. unsubstituted N-vinyllactams and N-vinyllactam derivatives, which can, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. and mixtures thereof.

Preferred monomers of formula (I) are those for which R² is CH₂═CH— and R¹ and R³ together with the amide group to which they are bonded form a lactame having 5 ring atoms.

In one embodiment of this invention, preference is given to using N-vinylpyrrolidone, N-vinylcaprolactam, N-vinylformamide, acrylamide or mixtures thereof, with N-vinyl pyrrolidone being most preferred.

In one embodiment of this invention, the at least one polymer is selected from polymers comprising vinylpyrrolidone as polymerized units, i.e. from vinylpyrrolidone homo- and copolymers

In one embodiment of this invention, the polymer comprises at least 90% by weight of vinylpyrrolidone. In another embodiment of this invention, the polymer comprises more than 99% by weight of vinylpyrrolidone.

In one embodiment of this invention, the at least one polymer is polyvinylpyrrolidone (PVP). In one preferred embodiment of this invention, the at least one polymer is selected from PVP with a molecular weight M_{w} of from 10,000 to 1,600,000, preferably from 10,000 to 100,000, more preferably from 10,000 to 60,000.

In one embodiment of this invention, the at least one polymer is selected from PVP with a molecular weight M_{w} of from 50,000 to 60,000 g/mol.

In another embodiment of this invention, the at least one polymer is selected from vinylpyrrolidone copolymers. In one embodiment of this invention, the at least one polymer is selected from vinylpyrrolidone copolymers with a molecular weight M_{w} of from 10,000 to 1,600,000, preferably from 10,000 to 100,000, more preferably from 10,000 to 60,000.

In another embodiment of this invention, the at least one polymer is selected from polysulfone (PSU), polyethersulfone (PES) and polyphenysulfone (PPSU).

In still another embodiment of this invention, the at least one polymer is selected from carbohydrates like e.g. cellulose, sucrose, chitosan.

In still another embodiment of this invention, the at least one polymer is selected from polyethers like e.g. polytetrahydrofurane, polyethylene oxide, polypropylene oxide.

In still another embodiment of this invention, the at least one polymer is selected from polymers comprising (meth)acrylates as polymerized units like e.g. PMMA.

In still another embodiment of this invention, the at least one polymer is selected from polymers comprising amino groups like e.g. polyvinylamine, polyethyleneimine, polyaniline. In still another embodiment of this invention, the at least one polymer is selected from polymers comprising vinyl ethers as polymerized units like e.g. poly vinyl methyl ether (PVME)

In still another embodiment of this invention, the at least one polymer is selected from polymers comprising vinyl carboxylates as polymerized units like e.g. poly vinylacetate (PVAc). In still another embodiment of this invention, the at least one polymer is selected from polymers comprising vinyl alcohol as polymerized units like e.g. poly vinylalcohol (PVOH) or partly hydrolyzed PVAc.

In one embodiment of this invention, the molecular weight M_{w} of the at least one polymer is at least 10,000 g/mol. In another embodiment of this invention, the molecular weight M_{w} of the at least one polymer is at most 1,000,000 g/mol.

In another embodiment of this invention, the molecular weight M_{w} of the at least one polymer is at least 50,000 g/mol. In another embodiment of this invention, the molecular weight M_{w} of the at least one polymer is at most 100,000 g/mol.

Another embodiment of this invention is a method of making a metal oxide nanocomposite according to this invention, said method comprising

step a) preparing a mixture comprising at least one precursor of said metal oxide, at least one substantially water-free liquid phase and at least one polymer;

step b) solvothermally treating the mixture of step a) at a temperature in the range of greater than 100° C. to 200° C.

Step a)

Step a) is the preparation of a mixture comprising at least one precursor of said metal oxide, at least one substantially water-free liquid phase and at least one polymer.

The at least one precursor of the metal oxide can be any material, that is at least partially soluble in the substantially water-free liquid phase and which can be transformed into the respective metal oxide by the solvothermal treatment according to step b). Suitable precursors of the metal oxide may be metal halides, acetates, sulfates or nitrates, sulphates, phosphates, acetylacetonates, perchlorates. The metal oxide precursors may either be the anhydrous compounds or the corresponding hydrates. Preferred precursors are halides, for example zinc chloride or titanium tetrachloride, acetates, for example zinc acetate, and nitrates, for example zinc nitrate. A particularly preferred precursor is zinc nitrate. In general, zinc nitrate and preferably any hydrate thereof like e.g. Zn(NO₃)₂*2H₂0, Zn(NO₃)₂*4 H₂0, Zn(NO₃)₂*6 H₂0, and Zn(NO₃)₂*9H₂0 are suitable zinc oxide precursors.

In one preferred embodiment of this invention, Zn(NO₃)₂*6 H₂O is used as zinc oxide precursor.

The substantially water-free liquid phase comprises less than 20% by weight, preferably less than 15% by weight and more preferably less than 10% by weight of water. In one embodiment of this invention, the substantially water-free liquid phase comprises less than 5% by weight of water. In another embodiment of this invention, the substantially water-free liquid phase comprises less than 2% by weight of water. In still another embodiment of this invention, the substantially water-free liquid phase comprises less than 1% by weight of water.

In one embodiment of this invention, the mixture of step a) comprises less than 20% by weight, preferably less than 10% by weight, more preferably less than 5% by weight and still more preferably less than 2% by weight of protic solvents like e.g. water or alcohols. In one embodiment of this invention, the mixture of step a) comprises from 0.1 to 2% by weight of water. In another embodiment of this invention, the mixture of step a) comprises from 0.3 to 1% by weight of water.

In one embodiment of this invention, additionally to the potentially present hydrate water of the metal oxide precursor, small amounts of protic solvents, preferably water, are added to the mixture of step a) preferably before the solvothermal treatment. In a preferred embodiment of this invention, water is added to the mixture so that the amount of added water is from 0.1 to 2.0 vol.-% of the resulting mixture. In another preferred embodiment of this invention, water is added to the mixture of step a) so that the amount of added water is from 0.5 to 1.5 vol.-% of the resulting mixture. In still another preferred embodiment of this invention, water is added to the mixture of step a) so that the amount of added water is from 0.5 to 1.0 vol.-% of the resulting mixture.

In one preferred embodiment of this invention, the substantially water-free liquid phase consists of or comprises a polar aprotic solvent.

In a preferred embodiment of this invention, the substantially water-free liquid phase consists of or comprises a solvent selected from ethers (like e.g. diethylether, tetrahydrofurane), carboxylic acid esters (like e.g. ethyl acetate), ketones like e.g. acetone, lactones like e.g. 4-butyrolactone, nitriles like e.g. acetonitrile, nitro compounds like e.g. nitro methane, tertiary carboxylic acid amides like e.g. dimethylformamide (DMF), urea derivates like e.g. tetramethylurea or N,N-dimethylpropyleneurea (DMPU), sulfoxides like e.g. dimethylsulfoxide (DMSO), and sulfones like e.g. sulfolane.

In one preferred embodiment of this invention, the substantially water-free liquid phase consists of or comprises DMF.

In another embodiment of this invention, the substantially water-free liquid phase consists of or comprises DMSO.

In a preferred embodiment of the invention, the mixture of step a) is a dispersion or a solution.

In a preferred embodiment of the invention, the mixture of step a) is prepared by dispersing and / or dissolving the at least one metal oxide precursor in the at least one substantially water-free liquid phase at first and thereafter adding the at least one polymer to the resulting dispersion / solution. The polymer can be added in the form of the pure polymer or in its dispersed or dissolved form. If the polymer is added in its dispersed or dissolved form, it is preferred to use substantially the same substantially water-free liquid phase as it was used for dispersing and / or dissolving the metal oxide precursor.

The mixture of step a) can also be prepared by preparing a dispersion and/or solution of the at least one polymer in the at least one substantially water-free liquid phase firstly and thereafter dispersing and / or dissolving the metal oxide precursor in the dispersion/solution of polymer and substantially water-free liquid phase.

The mixture of step a) can also be prepared by preparing a mixture of the at least one polymer and the metal oxide precursor at first and thereafter dissolving / dispersing that mixture in the substantially water-free liquid phase.

Concentration

In one embodiment of this invention, the concentration of the metal oxide precursor in the dispersion / solution, as calculated in the precursor's pure, i.e. without hydrate water, is at least 0.01, preferably at least 0.4 g/l and more preferably at least 1.0 g/l. In this embodiment of the invention, the metal oxide precursor concentration in the dispersion/solution, as calculated in the precursor's pure, i.e. without hydrate water, is at most 15 g/l, preferably at most 8 g/l.

In one embodiment of this invention, the polymer concentration in the dispersion/solution is at least 1 g/l, preferably at least 5 g/l, more preferably at least 10 g/l and at most 30 g/l, preferably at most 25 g/l and more preferably at most 20 g/l.

In one preferred embodiment of this invention, Zn(NO₃)₂*6 H₂O is selected as the zinc oxide precursor, polyvinylpyrrolidone is selected as the at least one polymer, DMF is selected as the substantially water-free liquid phase.

In this embodiment of the invention, the concentration of Zn(NO₃)₂*6 H₂O in the disper-sion/solution is, calculated as the hexahydrate form, preferably at least 3 g/l, more preferably at least 5 g/l, still more preferably at least 7 g/l and preferably at most 20 g/l, more preferably at most 15 g/l, still more preferably at most 10 g/l. In the same embodiment of the invention, the concentration of polyvinylpyrrolidone in the dispersion / solution is preferably at least 5 g/l, more preferably at least 10 g/l and preferably at most 25 g/l, more preferably at most 20 g/l.

If the polymer is selected from the polymers of formula (I), the ratio between the number of mol of carbonyl groups n(C═O) and the number of mol of Zinc ions n(Zn²⁺), i.e. n(C═O)/n(Zn²⁺), is preferably in the range of from 0.1 to 10, preferably from 1 to 7, more preferably from 2 to 5.

In one embodiment of this invention, the mixture of step a) comprises less than 5 weight-%, preferably less than 1 weight-%, more preferably less than 0.1 weight-%, still more preferably less than 0.01 weight-% of a Bronsted base. Most preferably, the mixture of step a) comprises substantially no Bronsted base.

Step b)

The term “solvothermally” as used in this invention refers to the treatment of the mixture prepared in step a) at a pressure above atmospheric pressure and a temperature which generally is significantly above 273 K, i.e. for example at least 323 K or more, sometimes even above the boiling point of the liquid phase at atmospheric pressure. The pressure generally is from 1 bar to 200 bar, preferably from 1.5 bar to 100 bar, and most preferably from 1.5 bar to 10 bar.

Generally, the temperature is higher than 100° C., preferably in the range between higher than 100° C. and 200° C. In one embodiment of this invention, the temperature is at least 110° C. In another embodiment of this invention, the temperature is at least 115° C. In another embodiment of this invention, the temperature is at least 120° C. In one preferred embodiment of this invention, the temperature is at most 150° C. In another preferred embodiment of this invention, the temperature is at most 140° C. In still another preferred embodiment of this invention, the temperature is at most 130° C. In one embodiment of the present invention, the solvothermal treatment of step b) is performed in a sealed autoclave.

Another embodiment of this invention is a method of making a metal oxide nanocomposite according to this invention, said method comprising

step a) preparing a mixture comprising at least one precursor of said metal oxide, at least one substantially water-free liquid phase and at least one polymer;

step b) subjecting the mixture of step a) to microwave irradiation. A suitable microwave irradiation would e.g. be 300 W for 10 minutes. A suitable apparatus is e.g. from CEM's microwave synthesis systems like e.g. Discover® Labmate.

Duration of Solvothermal Treatment

In one embodiment of this invention, the duration of the solvothermal treatment, i.e. the time during which the mixture according to step a) is stirred or agitated under elevated temperature and elevated pressure, is at least 10 minutes, preferably at least 30 minutes, more preferably at least 1 hour, still more preferably at least 2 hours and at most 48 hours, preferably at most 24 hours, more preferably at most 12 hours and still more preferably at most 3 hours.

Step b), i.e. the solvothermal treatment, is preferably terminated by naturally cooling the reaction mixture.

In one preferred embodiment of the invention, the metal oxide nanocomposite is separated from the liquid phase after termination of step b). Methods to separate solids from liquids like e.g. centrifugation, filtration, and rotary evaporation are well known in the art.

In another preferred embodiment of the invention, the metal oxide nanocomposite is subjected to one or more washing steps with an appropriate solvent additionally to the preceding separation. Appropriate solvents are e.g. the ones contained in the substantially water-free liquid phase as listed above and/or C₁-C₄-alcanols.

In a preferred embodiment of this invention, at least one metal oxide precursor is Zn(NO₃)₂*y H₂O, wherein y is selected from 2, 4, 6 or 9, preferably 6, at least one polymer is polyvinylpyrrolidone (PVP) with a molecular weight M_{w} in the range of from 40,000 to 70,000, preferably from 50,000 to 60,000, the substantially water-free liquid phase is or comprises dimethylformamide (DMF), the concentrations in solution are from 5 g/l to 10 g/l, preferably from 6 g/l to 9 g/l for Zn(NO₃)₂*y H₂O and from 5 g/l to 15 g/l, preferably from 8 g/l to 12 g/l for PVP, the temperature of the solvothermal treatment of step b) is in the range of from 110° C. to 150° C., preferably from 120° C. to 130° C. and the time of the solvothermal treatment is from 1 hour to 4 hours, preferably from 2 hours to 3 hours.

In one embodiment of this invention, the metal oxide nanocomposite particles substantially consist of 10-90 weight-% of polymer and 90-10 weight-% of metal oxide. In another embodiment of this invention, the metal oxide nanocomposite particles substantially consist of 40-80 weight-% of polymer and 60-20 weight-% of metal oxide. In still another embodiment of this invention, the metal oxide nanocomposite particles substantially consist of 50-70 weight-% of polymer and 50-30 weight-% of metal oxide. “Substantially consist of” means here, that the overall amount of components different from metal oxide and polymer is less than 10 weight-%, preferably less than 5 weight-%, more preferably less than 2 weight-%, still more preferably less than 1 weight-% of the metal oxide nanocomposite particles.

It was one objective of this invention to provide a method for the synthesis of metal oxide nanocomposites with a number average particle size in the range of from 80 nm to 400 nm and a narrow particle size distribution, i.e. an as homogeneous as possible particle size.

The monodispersity index (MDI) is a measure for the size distribution of the metal oxide nanocomposite particles. MDI values between 1 (value of 1 would mean identical size of all particles) and 0 are theoretically possible.

In one embodiment of this invention, said monodispersity index MDI is greater than 0.9 (90%). In another embodiment of this invention, said monodispersity index MDI is greater than 0.95 (95%).

In still another embodiment of this invention, said monodispersity index MDI is greater than 0.99 (99%).

Another embodiment of the present invention is a method of making a metal oxide nanocomposite as described before, wherein step b) is terminated when the desired number average particle size of the metal oxide nanocomposite is reached.

One way to find out when to terminate step b) is to beforehand perform a series of experiments where ingredients, concentrations, temperature and reaction time are systematically varied, and to determine the particle size of the resulting metal oxide nano-composite for each set of parameters. Such kind of experiment reveals the correlation between the reaction parameters (in particular reaction time, concentration of ingredients, reaction temperature) and the particle size.

Another embodiment of this invention are the metal oxide nanocomposite particles obtained by the methods of manufacture according to this invention.

Another embodiment of this invention are mixtures containing said metal oxide nano-composite particles obtained by the method according to this invention. Such mixtures are e.g. dispersions additionally containing at least a liquid phase and optionally further ingredients.

Preferred embodiments of the present invention are compositions containing said metal oxide nanocomposite particles. Such compositions are preferably selected from dispersion, emulsions (either creams or lotions), sticks, gels, sprays, clear lotions, or wipes or any other composition suitable for use in protecting skin and/or hair against sun damage. The dispersion or emulsion may be a water-in-oil (W/O) emulsion, or an oil-in-water (O/W) emulsion, or a multiple phase emulsion.

Another embodiment of this invention is a method for the protection of polymeric, in particular thermoplastic materials against damages caused by UV irradiation comprising incorporating the metal oxide nanocomposite according to this invention into such materials.

### EXAMPLES

The following examples shall describe details of this invention without being a limitation of any kind thereof.

Synthesis of Zinc Oxide Nanocomposite

Zinc nitrate hexahydrate (99% Fluka), Polyvinylpyyrolidone (PVP, Mw ca. 55,000, Aldrich) and 1,1-Dimethylformamide (DMF, 99%, Merck) were used without further purification. In all syntheses, zinc nitrate hexahydrate was first dissolved in DMF under vigorous stirring. The PVP was subsequently added under vigorous stirring. The resulting mixture was then transferred into a 250 mL PTFE liner of a stainless steel autoclave (DAB3, Berghof Instruments GmbH, Germany). The autoclave was then sealed and placed in a customized heating jacket on a standard laboratory heating plate. The temperature of the heating plate was set and the autoclave was heated for a defined time before being removed. The autoclave was then air cooled, opened and the product was transferred to a glass tube. The solid product was separated from the mother solution by centrifugation (Eppendorf 5415C, Heraeus Labofuge®400) and the solid washed with DMF (three washes) and absolute ethanol (three washes) through successive cycles of sedimentation and redispersion. Thereafter, the particles were dispersed in absolute ethanol and a stable suspension was obtained.

Characterization

The resulting suspensions were diluted to a suitable concentration and were examined by dynamic light scattering (Zetasizer®Nano, Malvern Instruments) and spectrophotometry (Cary®100 Scan, Varian). Samples for TEM and SEM were prepared by evaporating a droplet of suspension onto copper grids covered with a holey or continuous carbon film or on silicon wafer, respectively.

TEM was carried out on a Philips CM300 LaB6/UT instrument operating at 300 kV. SEM was carried out on a Zeiss ULTRA™ 55.

Image analysis was carried out by a threshold/watershed method using the ImageJ® package. The particles were modeled as ellipses with the diameter taken as the average of the major and minor axes. The dispersity of the ensemble's particle size is defined as the ratio between the mean number (or density) size distribution and the mean volume (or weight) size distribution. It can also be expressed as percentage (monodispersity index, MDI).

Effect of Temperature on Size and Polydispersity of ZnO Nanocomposite

The solvothermal reaction (step b) was carried out at two different temperatures, i.e. 125 and 150° C., the initial concentrations of metal oxide precursor being the same (volume of DMF 40 ml).

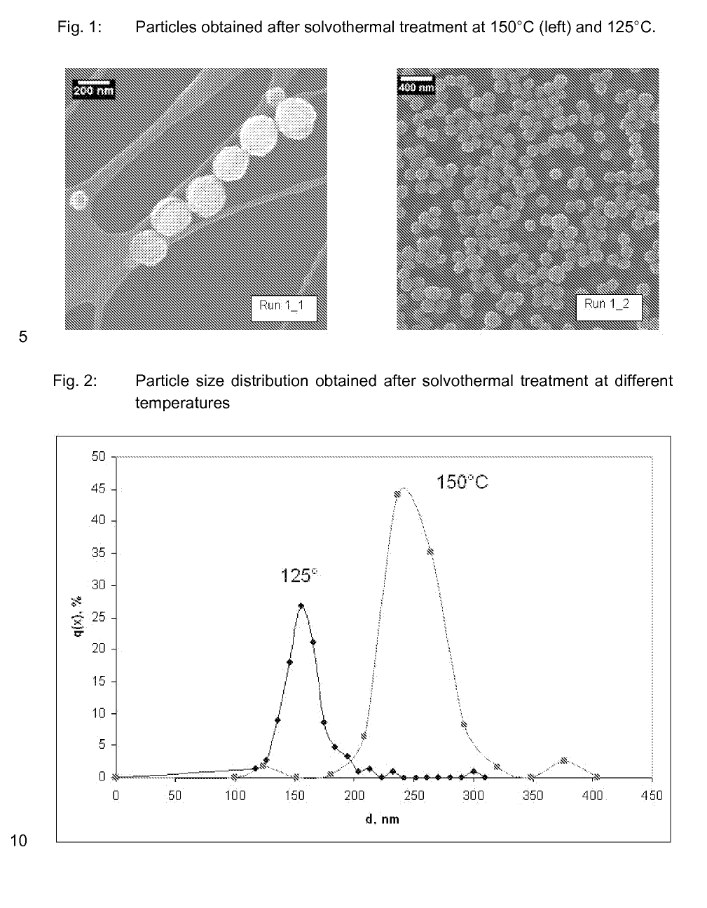

FIGS. 1 and 2 show that after the solvothermal treatment at 125° C. smaller particles and a more narrow particle size distribution (MDI about 99.4%) for the particles are received as compared to 150° C.

TABLE 1
Synthesis of ZnO nanocomposite: temperature

| Run | Zn(NO₃)₂6H₂O, g/L | PVP, g/L | T, ° C. | Time | MDI |
|---|---|---|---|---|---|
| 1_1 | 7.5 | 10 | 150 | 2 h 45 min | 93% |
| 1_2 | 7.5 | 10 | 125 | 2 h 45 min | 100% |

Effect of Concentration and Aging time on ZnO Nanocomposite

The concentration of the ZnO precursor (Zn(NO₃)₂*6H₂O) was increased from 7.5 g/l up to 15 g/l, while keeping constant reaction time (2 h 50 min), temperature (125° C.) and solvent volume (DMF, 40 ml).

In all experiments, the molar ratio n_{C═O}/n_{Zn2+} was kept constant at about 3.5 mol/mol by accordingly modifying the amount of PVP.

TABLE 2
effect of concentration and aging time

| Run | Zn(NO₃)₂*6H₂O, g/L | PVP, g/L | Time | |
|---|---|---|---|---|
| 2_1 | 7.5 | 10 | 2 | h 50 min |
| 2_2 | 7.5 | 10 | 24 | h |
| 2_3 | 15 | 20 | 2 | h 50 min |

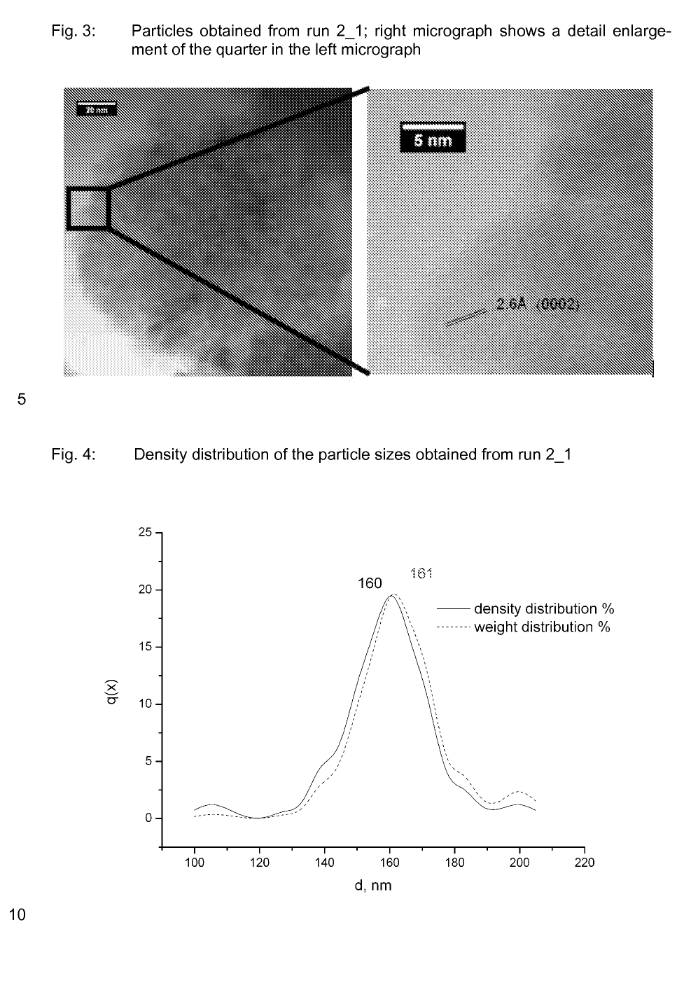

FIG. 3 shows Zinc oxide nanocomposite particles obtained from run 2_{—}1 comprising smaller interconnected subunits.

FIG. 4 shows the narrow particle size distribution of the nanocomposite obtained from run 2_{—}1, i.e. the high monodispersity of the ensemble of particles with respect to particle size.

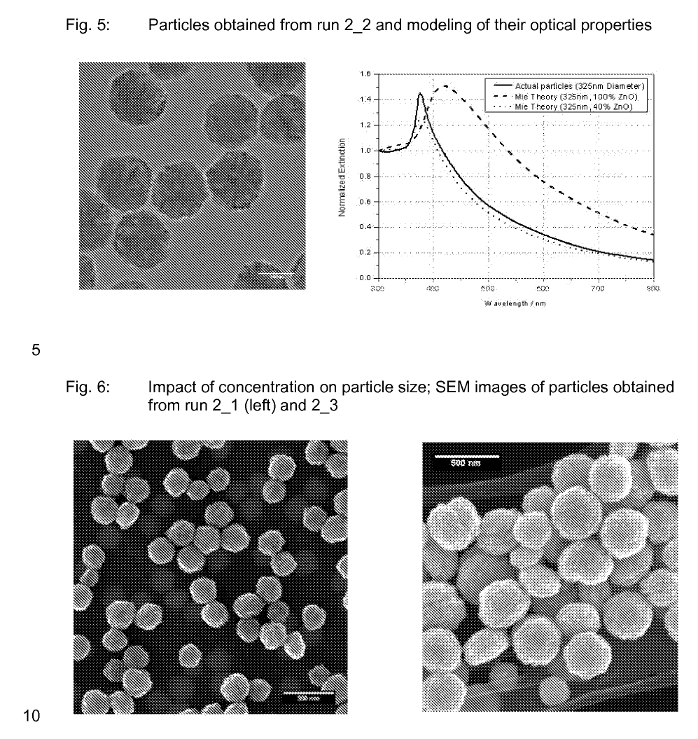

FIG. 5 shows the extinction spectrum of the sample of run 2_{—}2 as measured (solid line). Mie Theory allows the calculation of the extinction spectrum of a compact, pure ZnO sphere of the same size (325 nm). Mie theory provides an exact solution for spherical particles, for a given refractive index. Values for the wavelength-dependent refractive index for ZnO are taken from H. Yoshikawa, S. Adachi; Jpn. J. Appl. Phys. 36, 6237 (1997). Such calculated extinction spectrum of a compact, pure ZnO sphere is shown in FIG. 5 with a dashed line and can thus be compared to the spectrum of the nano-composite particles of the same size.

It can be seen, that the optical properties of the nanocomposite particles of this invention are superior for the use in e.g. transparent UV protection. While UV-A absorption (320-400 nm) is similar, transparency is significantly improved for the nanocomposite particles (i.e. reduced extinction of visible light, 400-800 nm) compared to simulated solid particles.

It can be seen that the measured spectrum of the nanocomposite particles of this invention can be simulated very well by the calculated spectrum of a spherical composite particle containing 40 weight % ZnO.

FIG. 6 shows that increasing the concentration of metal oxide precursor and polymer while keeping the reaction time constant leads to an increased mean size of the particles of about 382 nm with a MDI of 96%.

Effect of Added Water on the Synthesis of ZnO Nanocomposite

The addition of water to the reaction mixture of step a) has an impact on the size and the morphology of the metal oxide nanocomposites of this invention.

Different amounts of additional water were added to the mixture (the concentration of the starting solution was 15 g/l in Zn(NO₃)₂*6H₂O) and the reaction mixture was then solvothermally treated for 2h 50 min.

Table 3 reports the respective quantities used in these experiments. In all experiments the mol ratio n(C═O)/n(Zn²⁺) was kept constant to about 3.5 mol/mol.

TABLE 3
Synthesis of ZnO nanocomposite: effect of added water

| Run | Zn(NO₃)₂*6H₂O, g/L | PVP, g/L | H₂O, % (volume ratio) |
|---|---|---|---|
| 3_1 | 15 | 20 | — |
| 3_2 | 15 | 20 | 0.625 |
| 3_3 | 15 | 20 | 1.25 |
| 3_4 | 15 | 20 | 2.5 |

Increasing the amount of water in the reaction mixture of step a) leads to an increase of the size of the single ZnO subunits whereas the number average particle size remains nearly constant. The relative amount of metal oxide with respect to the amount of polymer in the nanocomposite particles increases with increased water content of the reaction mixture.

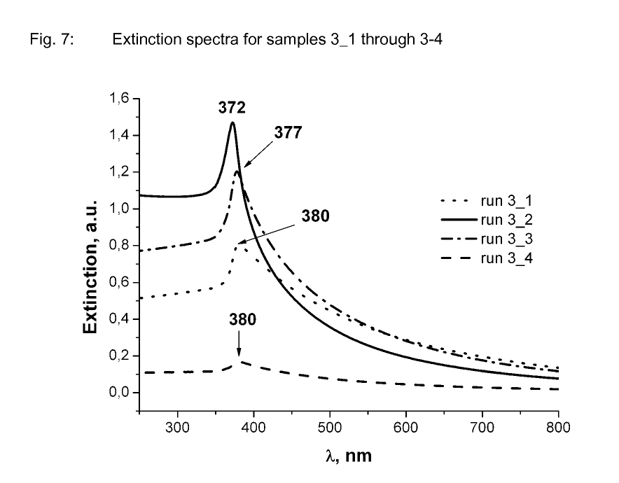

FIG. 7 shows the measured extinction spectra of samples 3_{—}1 through 3_{—}4. The optical properties with respect to transparency in the visible range and simultaneous effective UV protection (i.e. high extinction from 320 to 400 nm and simultaneously low extinction from 400 to 800 nm) are best for sample 3_{—}2.

Application Examples: Personal Care Formulations

General procedure for producing cosmetic preparations comprising zinc oxide nano-composites according to the invention

The respective phases A and C are heated separately to ca. 85° C. Phase C and zinc oxide nanocomposite are then stirred into phase A with homogenization. Following brief afterhomogenization, the emulsion is cooled to room temperature with stirring and topped up. All amounts are based on the total weight of the preparations. As Zinc oxide nanocomposite, the Zinc oxide according to Run 2_{—}1 is used. Of course, all other Metal oxides according to this invention can be used, in particular those of examples Run 2_{—}2, Run 2_{—}3, Run 3_{—}1, Run3_{—}2, Run 3_{—}3, run 3_{—}4.

### Example 1

Emulsion A, comprising 3% by weight of Uvinul® T150 and 4% by weight of zinc oxide nanocomposite according to the invention

| Phase | % by wt. | INCI |
|---|---|---|
| A | 8.00 | Dibutyl adipate |
| | 8.00 | C₁₂-C₁₅ alkyl benzoate |
| | 12.00 | Cocoglycerides |
| | 1.00 | Sodium cetearyl sulfate |
| | 4.00 | Lauryl glucoside, polyglyceryl-2 |
| | 2.00 | Cetearyl alcohol |
| | 3.00 | Ethylhexyl triazone (Uvinul ® T150) |
| | 1.00 | Tocopheryl acetate |
| B | 4.0 | Zinc oxide nanocomposite |
| C | 3.00 | Glycerin |
| | 0.20 | Allantoin |
| | 0.30 | Xanthan gum |
| | 0.02 | Triethanolamine |
| | ad 100 | Aqua dem. |

### Example 2

Emulsion B, comprising 3% by weight of Uvinul® T150, 2% by weight of Uvinul® A Plus and 4% by weight of Zinc oxide nanocomposite according to the invention

| Phase | % by wt. | INCI |
|---|---|---|
| A | 8.00 | Dibutyl adipate |
| | 8.00 | C12-C15 alkyl benzoate |
| | 12.00 | Cocoglycerides |
| | 1.00 | Sodium cetearyl sulfate |
| | 4.00 | Lauryl glucoside, polyglyceryl-2 |
| | 2.00 | Cetearyl alcohol |
| | 3.00 | Ethylhexyl triazone (Uvinul ® T150) |
| | 1.00 | Tocopheryl acetate |
| | 2.00 | Diethylamino hydroxybenzoyl hexyl benzoate |
| | | (Uvinul ® A Plus) |
| B | 4.0 | Zinc oxide nanocomposite |
| C | 3.00 | Glycerin |
| | 0.20 | Allantoin |
| | 0.30 | Xanthan gum |
| | 1.50 | Magnesium aluminum silicate |
| | ad 100 | Aqua dem. |

### Example 3

Emulsion A, comprising 3% by weight of Uvinul® T150 and 4% by weight of Zinc oxide nanocomposite according to the invention

| Phase | % by wt. | INCI |
|---|---|---|
| A | 8.00 | Dibutyl adipate |
| | 8.00 | C₁₂-C₁₅ alkyl benzoate |
| | 12.00 | Cocoglycerides |
| | 1.00 | Sodium cetearyl sulfate |
| | 4.00 | Lauryl glucoside, polyglyceryl-2 |
| | 2.00 | Cetearyl alcohol |
| | 3.00 | Ethylhexyl triazone (Uvinul ® T150) |
| | 1.00 | Tocopheryl acetate |
| B | 4.0 | Zinc oxide nanocomposite |
| C | 3.00 | Glycerin |
| | 0.20 | Allantoin |
| | 0.30 | Xanthan gum |
| | 0.02 | Triethanolamine |
| | ad 100 | Aqua dem. |

### Example 4

Emulsion B, comprising 3% by weight of Uvinul® T150, 2% by weight of Uvinul® A Plus and 4% by weight of Zinc oxide nanocomposite according to the invention

| Phase | % by wt. | INCI |
|---|---|---|
| A | 8.00 | Dibutyl adipate |
| | 8.00 | C12-C15 alkyl benzoate |
| | 12.00 | Cocoglycerides |
| | 1.00 | Sodium cetearyl sulfate |
| | 4.00 | Lauryl glucoside, polyglyceryl-2 |
| | 2.00 | Cetearyl alcohol |
| | 3.00 | Ethylhexyl triazone (Uvinul ® T150) |
| | 1.00 | Tocopheryl acetate |
| | 2.00 | Diethylamino hydroxybenzoyl hexyl benzoate |
| | | (Uvinul ® A Plus) |
| B | 4.0 | Zinc oxide nanocomposite |
| C | 3.00 | Glycerin |
| | 0.20 | Allantoin |
| | 0.30 | Xanthan gum |
| | 1.50 | Magnesium aluminum silicate |
| | ad 100 | Aqua dem. |

### Example 5

| Phase | % by wt. | Constituents | INCI |
|---|---|---|---|
| A | 7.50 | Uvinul ®MC 80 | Ethylhexyl methoxycinnamate |
| | 1.50 | Tween ®20 | Polysorbate-20 |
| | 3.00 | Pationic ®138 C | Sodium lauroyl lactylate |
| | 1.00 | Cremophor ®CO 40 | PEG-40 hydrogenated castor oil |
| | 1.00 | Cetiol ®SB 45 | Butyrospermum Parkii |
| | | | (Shea Butter) |
| | 6.50 | Finsolv ®TN | C12-15 alkyl benzoate |
| B | 5.00 | Zinc oxide | |
| | | nanocomposite | |
| C | 1.00 | D-Panthenol 50 P | Panthenol, propylene glycol |
| | 4.00 | 1,2-Propanediol | 1,2-Propanediol |
| | 0.30 | Keltrol ® | Xanthan gum |
| | 0.10 | Edeta ®BD | Disodium EDTA |
| | 2.00 | Urea | Urea |
| | 2.00 | Simulgel ®NS | Hydroxyethyl acrylate/sodium |
| | | | acryloyldimethyl taurate |
| | | | copolymer, squalane, polysor- |
| | | | bate 60 |
| | 64.10 | Water dem. | Aqua dem. |
| D | 0.50 | Lactic acid | Lactic acid |
| | 0.50 | Euxyl ®K 300 | Phenoxyethanol, methylparaben, |
| | | | butylparaben, ethylparaben, |
| | | | propylparaben, isobutylparaben |

Phase A was heated to 80° C., then phase B was added, the mixture was homogenized for 3 minutes. Phase C was heated separately to 80° C. and stirred into the mixture of phases A and B. The mixture was then cooled to 40° C. with stirring, then phase D was added. The lotion was briefly afterhomogenized.

### Example 6

Water-in-Silicone Formulation

| % by wt. | Ingredients | INCI |
|---|---|---|
| Phase A | | |
| 25.0 | Dow Corning 345 Fluid | Cyclopentasiloxane, |
| | | cyclohexasiloxane |
| 20.0 | Luvitol ™ Lite | Cyclopentasiloxane |
| 8.0 | Uvinul ® MC 80 | Ethylhexyl methoxy- |
| | | cinnamate |
| 4.0 | Abil ® EM 90 | Cetyl PEG/PPG-10/1 |
| | | dimethicone |
| 7.0 | T-Lite ™ SF | Titanium dioxide (and) |
| | | aluminum hydroxide (and) |
| | | dimethicone/methicone |
| | | copolymer |
| Phase B | | |
| 17.0 | Ethanol 95% | Alcohol |
| 5.0 | Zinc oxide nanocomposite | |
| 4.0 | 1,2-Propanediol | 1,2-Propanediol |
| 5.0 | Water dem. | Aqua dem. |
| 3.0 | Glycerol 87% | Glycerin |
| 1.0 | Talc (C/2S, Bassermann) | Talc |

Phases A and B are homogenized at ca. 11 000 rpm for 3 minutes, then B is added to A and homogenized for another minute.

### Example 7

| | |
|---|---|
| A (% by wt.) | |
| 7.00 Uvinul ®MC 80 | Ethylhexyl methoxycinnamate |
| 2.00 Uvinul ®A Plus | Dimethylamino hydroxybenzoyl |
| | hexyl benzoate |
| 5.00 Uvinul ®N 539 T | Octocrylene |
| 3.00 Octyl salicylate | Octyl salicylate |
| 3.00 Homomenthyl salicylate | Homosalate |
| 2.00 Antaron ®V-216 | PVP/hexadecene copolymer |
| 0.50 Abil ®350 | Dimethicone |
| 0.10 Oxynex ®2004 | BHT, ascorbyl palmitate, |
| | citric acid, glyceryl stearate, |
| | propylene glycol |
| 2.00 Cetyl alcohol | Cetyl alcohol |
| 2.00 Amphisol ®K | Potassium cetyl phosphate |
| B | |
| 3.00 Zinc oxide nanocomposite | |
| 5.00 1,2-Propylene glycol Care | Propylene glycol |
| 57.62 Water | Aqua dem. |
| 0.20 Carbopol ®934 | Carbomer |
| 5.00 Witconol ®APM | PPG-3 myristyl ether |
| C | |
| 0.50 Euxyl ®K300 | Phenoxyethanol, methylparaben, |
| | ethylparaben, ethylparaben, |
| | butylparaben, propylparaben and |
| | isobutylparaben |

Preparation:

Phase A is heated to melting at ca. 80° C. and homogenized for ca. 3 min; phase B is-likewise heated up to ca. 80° C., added to phase A and this mixture is homogenized again. It is then left to cool to room temperature with stirring. Phase C is then added and the mixture is homogenized again.

1. A method of protecting an object against UV radiation comprising applying to said object an effective amount of a composition containing a metal oxide nanocomposite, said metal oxide nanocomposite
a) having a number average particle size in the range of from 80 nm to 400 nm, and
b) comprising at least one metal oxide and at least one polymer, and
c) being substantially in the form of interconnected metal oxide units dispersed in a matrix substantially consisting of the at least one polymer.

2. The method according to claim 1, wherein the at least one metal oxide is Zinc oxide.

3. The method according to claim 1, wherein the at least one polymer is selected from polymers comprising N-vinylpyrrolidone as polymerized units.

4. The method according to claim 1, wherein the at number average particle size of the metal oxide nanocomposite is in the range of from 100 nm to 200 nm.

5. A method of making a metal oxide nanocomposite as defined in claim 1, the method comprising
a) preparing a mixture comprising at least one precursor of said metal oxide, at least one substantially water-free liquid phase and at least one polymer;
b) solvothermally treating the mixture of step a) at a temperature in the range of greater than 100° C. to 200° C.

6. The method of making a metal oxide nanocomposite according to claim 5, wherein the temperature in step b) is in the range of from 110° C. to 150° C.

7. The method of making a metal oxide nanocomposite according to claim 5, wherein the mixture of step a) is a dispersion or a solution.

8. The method of making a metal oxide nanocomposite according to claim 5, wherein the metal oxide is Zinc oxide.

9. The method of making a metal oxide nanocomposite according to claim 5, wherein the at least one substantially water-free liquid phase is or comprises a polar aprotic solvent.

10. The method of making a metal oxide nanocomposite according to claim 9, wherein the polar aprotic solvent is N,N-dimethylformamide.

11. The method of making a metal oxide nanocomposite according to claim 5, wherein step b) is terminated when the desired number average particle size of the metal oxide nanocomposite is reached.

12. A metal oxide nanocomposite obtainable by a method according to claim 5.

13. The method of claim 1, wherein the object comprises human skin.
